(19) United States
(12) Patent Application Publication
(10) Pub. No.: US 2012/0045426 A1
(43) Pub. Date: Feb. 23, 2012

(54) COMPOSITIONS FOR REDUCING THE DELETERIOUS EFFECTS OF STRESS AND AGING

(76) Inventors: John A. St. Cyr, Minneapolis, MN (US); Dean J. MacCarter, Englewood, CO (US)

(21) Appl. No.: 13/214,878

(22) Filed: Aug. 22, 2011

Related U.S. Application Data
(60) Provisional application No. 61/401,872, filed on Aug. 21, 2010.

(51) Int. Cl. A61K 38/48; A61P 9/00; A61P 7/00; A61P 39/06

(52) U.S. Cl. 424/94.64

(57) ABSTRACT

The invention provides a formulation for treating stress and lessening fatigue. The formulation can be combined with water or another suitable liquid to provide a beverage for ease of administration. The formulation can include one or more of an energy compound, a vasodilator, a vasodilator adjuvant, and an antioxidant enhancer. In a typical formulation the energy compound is D-ribose or guanosine. The formulation can improve energy and alertness, and reduce the effects of stress and fatigue.

### RELATED APPLICATIONS

This application claims priority under 35 U.S.C. §119(e) to U.S. Provisional Patent Application No. 61/401,872, filed Aug. 21, 2010, which is incorporated herein by reference.

### BACKGROUND OF THE INVENTION

Many individuals turn to dietary supplements to enhance their daily energy, to decrease fatigue, or to maintain or increase their productivity. A popular approach is to use a so-called “energy drink.” The most common stimulants in such energy drinks is caffeine or a caffeine-containing herb. Other common ingredients in energy drinks include guarana, verba mate, acai, taurine, ginseng, maltodextrin, inositol, L-carnitine, creatine, glucuronolactone, gingko biloba and carbohydrates such as sucrose and high fructose corn syrup. “Diet” energy drinks replace the carbohydrates with artificial sweeteners. These drinks can produce higher levels of alertness and perceived energy. However, the caffeine levels in these drinks are generally so high that they cause adverse effects such as irritability, insomnia, and agitation, sometimes referred to as “coffee nerves” or “the jitters.” Many users also experience a “let down” period or “crash” following a stimulation interval, which in itself causes negative physiological effects. Furthermore, many of these drinks contain an excessive amount of refined carbohydrates, which additionally taxes the body's regulatory systems.

The stimulatory effect is seen at the cellular level as the production of reactive oxygen species (ROS), low cellular energy levels, oxygen debt, and elevation of the neurotransmitter dopamine. The average cup of coffee contains 40 milligrams of caffeine, and the average energy drink contains about 150 milligrams. Toxic levels can be achieved by drinking more than one serving. The United States and the European Union are considering banning high caffeine beverages, especially those coupled with alcohol. The United Kingdom already prohibits sale of the popular Red Bull energy drink to children under 16 years of age, and several deaths associated with beverages containing caffeine and alcohol have been reported. Furthermore, these beverages are stimulatory only and do not address other factors involved in fatigue, thereby leading to an imbalance in cellular metabolism and a loss of homeostasis.

This imbalance in cellular metabolism and loss of homeostasis may be due to oxygen debt, that is, insufficient oxygen at the cellular level or to the formation of free radicals during normal, low or high levels of oxygen. Free radicals are highly reactive and can damage cell membranes, interfere with protein synthesis, and even cause cell death. Peroxides and hydroxyl radicals are formed during normal metabolic processes, in particular, during mitochondrial oxidative phosphorylation, where oxygen as the terminal electron acceptor forms a superoxide anion. It is estimated that about 2 to 5% of the total oxygen uptake during both rest and exercise has the ability to form superoxide anions. When exercise or stress increases mitochondrial activity, production of ROS increases. The increased presence of ROS also occurs with aging, as mitochondrial efficiency declines, and the stimulatory effect of caffeine further increase the production of ROS.

Accordingly, there is a need for compositions that avoid high levels of stimulants such as caffeine and excessive carbohydrates. There is also a need for compositions, such as beverages, that that reduce ROS formation, thereby relieving fatigue and restoring homeostasis to persons experiencing stress or aging.

### SUMMARY

The invention provides a formulation that includes two or more of an energy compound, a vasodilator, a vasodilator adjuvant, and an antioxidant enhancer. In one embodiment, the composition includes an energy compound and at least one of a vasodilator, a vasodilator adjuvant, and an antioxidant enhancer. The energy compound can be, for example, D-ribose or guanosine, creatine, L-carnitine, or a combination thereof.

In one embodiment, the antioxidant enhancer is carnosine, anserine, coffeeberry extract, cocoa bean extract, or a combination thereof.

In some embodiments, the vasodilator is citrulline, L-arginine, nitroglycerin, papaverine, isoproterenol, nylidrin, isoxsupine, nitroprusside, dippyramide, hydralazine, minoxidol, diazoxide, adenosine, xanthine, ethyl alcohol, a nitrate (e.g., NaNO₃ or KNO₃), a nitrite (e.g., NaNO₂ or KNO₂), or a combination thereof.

In some embodiments, the vasodilator adjuvant is nattokinase, aspirin, coumadin, or a combination thereof. The composition can include one, two, three, or four different antioxidant enhancers. For example, a first vasodilator adjuvant can be nattokinase, and one or more additional antioxidant enhancers can be anserine, coffeeberry powder, or cocoa bean powder.

In one embodiment, the wt.% ratio of the energy compound to the antioxidant enhancer is about 1.6:1 to about 7.5:1. In some embodiments, the wt.% ratio of the energy compound to the vasodilator is about 2:1 to about 6:1.

Any of the compositions described herein can be combined with a liquid to form a beverage. Examples of suitable liquids include water, sparkling water, fruit juice, tea, coffee, hot chocolate, and the like.

One specific composition includes a) D-ribose or guanosine; b) citrulline or L-arginine; c) nattokinase; and d) carnosine. The composition can further include anserine, coffee berry powder, cocoa bean powder, or a combination thereof.

The invention thus provides methods for treating stress and fatigue comprising administering to a human in need of such treatment an effective amount of a composition as described herein, thereby treating the stress and/or fatigue.

The invention also provides a method to treat oxidative stress comprising administering to a human experiencing oxidative stress an effective amount of a composition as described herein, thereby reducing the amount of oxidative stress experienced by the human.

The invention further provides a method to increase tissue perfusion comprising administering to a human in need of increased tissue perfusion an effective amount of a composition as described herein, thereby increasing the tissue perfusion in the human.

The invention yet further provides a method to aid aerobic work performance comprising administering to a human engaging in aerobic work an effective amount of a composition as described herein, thereby increasing the aerobic work capacity of the human.

The invention additionally provides a method to improve blood viscosity comprising administering to a human an effective amount of a composition as described herein, thereby reducing the viscosity of the blood in the human, for example, as determined by a blood viscometer (for example, a Hemathix blood analyzer, model SCV-200, Health Onvector, Inc.), optionally in combination with monitoring capillary blood flow, for example, using infrared monitoring and a dermal patch placed over the exercising muscle bundle, whereby enhanced oxygen delivery to the exercising muscle can be observed. Improving (e.g., reducing) blood viscosity can also alleviate the effects of certain environmental conditions, such as encountering high altitudes or pressurized states with reduced oxygen content. Consumption of a composition as described herein can also reduce the occurrence or severity of sickle cell anemia or other red blood cell malformations, for example, by reducing blood viscosity.

In some embodiments, the formulation or composition can exclude certain ingredients such as sucrose or caffeine. In other embodiments, the formulation will include less than about 7 wt. %, less than about 5 wt. %, less than about 3 wt. %, less than about 2 wt. %, less than about 1.5 wt. %, less than about 1 wt. %, less than about 0.5 wt. %, less than about 0.1 wt. %, of sucrose and/or caffeine. In some embodiments, the formulation includes less than about 100 mg, less than about 75 mg, less than about 50 mg, less than about 25 mg, less than about 10 mg, less than about 5 mg, less than about 2 mg, or less than about 1 mg of sucrose and/or caffeine. Such limitations can also be extended to sweeteners such as high fructose corn syrup.

The invention also provides for the use of the compositions described herein for use in medical therapy. The medical therapy can be lessening the untoward effects of stress, for example, for treating fatigue and neutralizing the formation of ROS. The invention provides for the use of the compositions described herein for the manufacture of medicaments useful for the treatment of fatigue or stress in a mammal, such as a human. The medicament can include a pharmaceutically acceptable diluent, excipient, or carrier.

### DETAILED DESCRIPTION

Fatigue. Fatigue, or low perceived energy levels, is associated with various conditions such as exertion, inanition, or lack of sleep; an imbalanced or inappropriate diet; acute or chronic stressful states; and can be a concomitant of aging. The degree of an individual's fatigue varies with the causative factors and the duration they have been present. There is also a variance in how each individual person deals with or tolerates fatigue. Intense fatigue can produce physical and/or mental symptoms, negatively affecting one's abilities both during wakefulness and sleep.

Fatigue is routinely assessed subjectively because quantifying the precise state of fatigue is difficult. Objective means of assessing fatigue have only recently been developed. These objective means include measuring the status of the cardiac-pulmonary axis, measuring perfusion and blood supply to the tissues, and analyzing urinary metabolites for ROS. Subjective and objective assessments together are therefore powerful tools that can assess and/or diagnose a potentially dangerous condition. Reducing or eliminating the causative factors, along with elucidating ways to resolve fatigue states are important steps to manage this condition. However, appropriately managing fatigue is not always easy because identifying causative stressors can be difficult and the aging process is unavoidable.

Some type of stress is present nearly every moment of every day in the average individual and every individual deals with stress differently. The spectrum of stress ranges from mild irritation to more severe forms that can seriously affect an individual's health. Too much stress can have both acute and long lasting ramifications. The psychological and physiological reactions to stress can be mild or severe. However, these reactions tax the cellular energy balance and cellular vitality.

Among the symptoms associated with stress due to a state of alarm include adrenaline production, muscular tension, short term resistance as a coping mechanism, exhaustion, degrees of acute and persistent fatigue, irritability, inability to concentrate, and physiological effects such as elevated heart rate and blood pressure. In an acute episode and definitely in the prolonged, chronic state of stress, bodily functions are often altered. Stress can affect the immune system, the neuro-endocrine axis, reasoning ability, concentration and other measurable parameters of cognition, and cause cardiopulmonary instability. Common physically symptoms such as aches/pains, fatigue or a “lack of energy”, gastrointestinal symptoms, a lack of appetite, lightheadedness, dizziness, tachycardia, chest discomfort, insomnia, and a prevalence of colds due to an altered immune system. Mental effects can include experiencing concentration and memory problems, poor judgment, anxiousness, a jittery sensation with a state of chronic worrying, each of which can result in a vicious cycle of increasing stress. Mere stimulation to increase energy does not deal with the effects of stress and, in fact, may acerbate the condition.

Fatigue from stress is difficult to treat. Many individuals search the literature to better understand fatigue from stress to find ways to deal with their stress-related symptoms. Numerous research articles address the use of nutraceuticals and/or supplements that may offer benefits in lessening symptoms associated with stress and fatigue due to aging. These articles often focus on the decrease in cellular energy, the production of free radicals including reactive oxygen species (ROS) and oxygen debt. Addressing these problems with nutraceuticals and/or supplements requires a strategic design. Development of such should be based on sound scientific principles and data to support significant claims, rather than mere theory.

In addition to those experiencing the adverse effects of stress, the aging population worldwide continues to grow due to longer life expectancies, improved healthcare, and increased attention to diet. However, with aging come physiological and cognitive changes, such as decreasing vision and hearing, decreasing muscular strength, and reduced energy production, which factors result in the common complaint of fatigue. Commonly, with this developed state of fatigue, there is decreased desire to exercise, resulting in weight gain that can contribute to the development of a state of overweight or obesity, which has been shown to be an important factor in many illnesses. With aging, maximum oxygen uptake and heart rate depart from normal guidelines. To maintain energy levels, individuals may need to rely on other mechanisms to improve oxygen and nutrient delivery to exercising muscle. Vascular function may be compromised resulting in reduced blood flow through muscle capillary beds during rest as well as during exercise.

Fatigue is a broad descriptive term that encompasses feelings of tiredness, drowsiness, lethargy, malaise, weakness, and lack of energy. In fact, approximately 20% of Americans complain of fatigue so intense that it interferes with their daily lives, causing them to seek therapy to regain lost energy and to feel less tired or stressed.

Aging. Several worldwide conferences dedicated to aging have sought a better understanding of the aging process. Discussion topics have included reports on investigation into developing better life styles with therapeutic regimens culled from pertinent scientific studies. For example, at a conference sponsored by the Scripps Center for Integrative Medicine in 2009 (MacCarter D, Flannigan R, Washam M, Shecterle LM, St.Cyr J.; “D-ribose improves fatigue in adults.” J. Alt. & Comp. Med. 15(7):812, 2009), the benefits of oral D-ribose for lessening physical fatigue and improving mental outlook in aging individuals (>50 years of age), who complained initially of tiredness for at least one month, was reported. Objectively, the study subjects found improvement in aerobic fitness, breathing efficiency, oxygen uptake efficiency, heart rate to oxygen uptake coupling and metabolic energy expenditure. Subjectively, they experienced a significant improvement in a mental abilities and vitality. In part, this study showed that as one ages, there is the need to recapture or slow the rate of these physical and mental changes. Many of these publications describe the role of nutraceuticals and/or supplements in providing insight into dealing with the many physical and mental changes that occur during aging.

### Perfusion and Homeostasis

Conditions such as fatigue and stress vary in expression from mild in a healthy person not subjected to high levels of stress to extreme. Extreme fatigue and stress can contribute to disease and even death in certain populations, particularly the elderly. The compositions described herein are beneficial to the entire range of these populations. Even persons not chronically fatigued or stressed will often encounter situations that cause transitory stress. Accordingly, such individuals can benefit from compositions that reduce stress and improve homeostasis.

Enhancing cellular metabolism is more complex than merely raising energy levels through ingestion of stimulants such as caffeine. However, the compositions described herein address numerous aspects of fatigue by, among other things, reducing the effect of ROS and relieving oxygen debt.

Adequate perfusion of tissue beds at all times, but especially during times of stress, is important to provide essential nutrients to the tissues, to minimize oxygen debt, and to maintain homeostasis. Stressful conditions can alter this normal network resulting in impaired cellular metabolism. It is therefore critical that adequate tissue perfusion be achieved for any substance recommended to be consumed during times of stress, or to be consumed prophylactically when stress is likely to be encountered, such as when initially embarking upon exercise routine. Furthermore, changes in perfusion can occur during aging as a result of vascular disease or partitioning of specific tissues. Enhanced cellular metabolism with improved perfusion is also of importance to maximize energy and minimize untoward symptoms. The compositions and methods described herein address many of these issues and concerns. These products are tailored to address the fatigue and tired state commonly found with aging. The compositions are also useful for persons who do not habitually encountering stress to prophylactically reduce ROS production.

### Metabolic Requirements

All cells require adequate energy levels of adenosine triphosphate (ATP) to maintain integrity and function. Normally, this demand and supply is met through adequate, balanced nutrition. However, under certain conditions (various aging and disease states) the demand is greater than the production of ATP. For example, during times of stress, these energy levels decrease and with continued stress there is a further depletion in energy compounds.

Individuals are less able to cope with day to day situations effectively with decreased energy levels, which can produce even more stress. Even in the aging population, the experience of fatigue may potentially reflect a mismatch in energy demand and supply, as a result of insufficient vascular blood flow to deliver an adequate amount of substrates and oxygen to the tissue beds.

Muscle tissue perfusion must be maintained in the exercise state and during recovery to not only generate the appropriate balance in energy supply and demand, but also to aid in replenishing the oxygen deficit that has occurred during higher intensities of exercise. For these reasons, many turn to advertised products that purportedly provide energy benefits. However, many of these “energy drinks” or “energy supplements” do not fulfill their lofty claims; and therefore, the product loses acceptance and credibility. Products that promote energy benefits, less fatigue, and a better well-being must have scientific data to support these claims to remain commercially viable.

Accordingly, there is a need for compositions increase tissue perfusion and help restore or maintain homeostasis. Such compositions should avoid high levels of stimulants such as caffeine and excessive carbohydrates. Described below are compositions, which can be in the form of beverages, that that reduce ROS formation, thereby relieving fatigue and restoring homeostasis to persons experiencing stress or aging.

### Definitions

For convenience, certain terms used in the specification and appended claims are collected and described herein below. As used herein, the recited terms have the following meanings All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001, or Webster's New World Medical Dictionary, 3^{rd} Edition, Wiley Publishing, Inc., 2008.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more can refer to one to five, or one to four, or one, two, three, four, five, six, or more.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

An “effective amount” refers to an amount effective to treat a disease, disorder, and/or condition, or to bring about a recited effect. For example, an amount effective can be an amount effective to reduce the progression or severity of the condition or symptoms being treated, or to improve or enhance a physiological parameter. Determination of an effective amount is well within the capacity of persons skilled in the art. The term “effective amount” is intended to include an amount of a formulation or composition described herein, or an amount of a combination of compounds described herein, e.g., that is effective to improve or enhance a physiological parameter, to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment, contacting, or administration.

“Energy compounds” include compounds directly or indirectly contributing to an increase of ATP concentration in the body. These compounds include glucose and other saccharides that are readily converted to glucose. Specific examples of energy compounds are D-ribose and guanosine. Creatine and L-carnitine can enhance the supply of ATP and aid the transportation of fatty acids for the production of energy compounds, respectively.

A “vasodilator” is a compound that causes vascular dilation of the arterioles of the capillary bed, thereby increasing perfusion of the area served by the capillary bed thereby carrying nutrients and oxygen to the tissue and removing breakdown products and CO₂. Vasodilators include drugs such as nitroglycerin, papaverine, isoproterenol, nylidrin, isoxsupine, nitroprusside, dippyramide, hydralazine, minoxidol and diazoxide and the naturally occurring compounds L-arginine, adenosine, xanthine, ethyl alcohol, and citrulline. Other vasodilators include nitrates (e.g., NaNO₃ or KNO₃) and nitrites (e.g., NaNO₂ or KNO₂).

“Vasodilator adjuvant” means a compound which, while not directly affecting vasodilation, increases the effect of vasodilation by decreasing the viscosity of blood. These compounds include aspirin, coumadin and nattokinase.

An “antioxidant” is a compound that neutralizes a reactive oxygen species (ROS), for example, when in the presence of the ROS in a mammalian organism. Neutralizing antioxidants include beta-carotene, coenzyme Q, selenium, vitamin C, and vitamin E, as well as acai, quercetin, pomegranate, astaxanthinor, or extracts thereof. These antioxidants react with and detoxify ROS.

“Antioxidant enhancers” are compounds that enhance the activity of an antioxidant in the body but no not necessarily possess antioxidant activity themselves. Antioxidant enhancers do not directly react with and detoxify ROS, but rather by stimulating alternative pathways, reduce the production of ROS. Antioxidant enhancers include carnosine, anserine, coffeeberry extract, and cocoa bean extract.

The term “perfusion” refers to the process of nutritive delivery of arterial blood to a capillary bed in biological tissues. Perfusion can be measured by subjective methods such as evaluating skin color, temperature, condition and capillary refill. Objective methods to measure perfusion include magnetic resonance imaging (MRI) techniques with contrast agent injection or arterial spin labeling.

“Ventilatory efficiency” (VE _{eff}) is defined as the linear slope of the regression plot of volume of ventilation in liters per minute on the “y” axis to liters of CO₂ exhaled on the “x” axis reflecting the ratio between breathing volume and elimination of CO₂ through expired air. It is commonly expressed as the linear slope of VE to VCO₂, VCO₂, being on the x-axis. Ventilatory efficiency (VE_{eff}) reflects the actual condition of the lungs, when measured during exercise (see Principles of Exercise Testing and Interpretation, 4^{th} Ed., Wasserman et al., Lippincott Williams & Wilkins, Philadelphia; pages 92-96, incorporated herein by reference). Ventilatory efficiency (VE_{eff}) is determined by the linear, sub-max or peak attained relationship between Minute Ventilation (VE) and carbon dioxide output (VCO₂), VE being on the “y axis” and the linear slope being determined using the linear regression model, y=a+bx, “b” representing the slope. The steeper the VE_{eff} slope, the worse the ventilation efficiency of the patient. VE_{eff} and methods for determining VE_{eff} are further described in U.S. Patent Publication No. 2010/0099630 (MacCarter et al.).

The VO₂ (volume of oxygen consumption) uptake efficiency is the linear slope of O₂ uptake per unit time plotted against the log of VE on the “x” axis. Oxygen pulse (O₂P) is a surrogate measurement of the heart stroke volume and is calculated by dividing VO₂ in mL per minute by the heart rate at that time.

### D-Ribose:

D-ribose, a natural occurring pentose carbohydrate, plays an important role in the production of adenosine triphosphate (ATP). D-ribose has been shown to enhance recovery from low levels of ATP in healthy exercising individuals, as well as to lessen the symptoms of individuals afflicted with fibromyalgia/chronic fatigue syndrome. More recently, the daily use of D-ribose in aging populations, such as the “Baby Boomers”, reduced both the physical and perceived cognitive symptoms of fatigue. D-ribose may also offer benefits in enhancing energy recovery with an improvement in energy stores, a lessening of fatigue and its accompanying symptoms, and a more satisfactory tolerable state during times of stress. Ribose also acts in the recycling of ATP and in so doing bypasses pathways that produce ROS, thus acting as an antioxidant enhancer.

An effective dose of D-ribose is about 0.5 g/dose to about 7 g/dose, depending upon the indication. A highly effective dose can be 1.5 g to about 3 g per dose, twice a day. A highly beneficial regimen is a daily dose totaling about 6 g, administered in two to four portions. Any dose of D-ribose can show beneficial effect, but the lower doses must be administered more times per day for maximal effect. Higher daily doses (e.g., greater than about 3 g or 6 g should be divided into more than one dose. D-ribose is available from many nutraceutical companies, such as Heartland Biosciences, Inc., Minneapolis, Minn.

### Guanosine:

Guanosine, a purine nucleoside, comprises guanine attached to a ribose ring via a β-N₉-glycosidic bond. Guanosine can be phosphorylated to become guanosine monophosphate (GMP), cyclic guanosine monophosphate (cGMP), guanosine diphosphate GDP), and guanosine triphosphate (GTP). Guanosine triphosphate aids in energy transfer within the cell. An effective dose of guanosine monophosphate can range between 500-1500 mg/dose. Guanosine monophosphate can be purchased from Hangzhou Dayanghen Co., Ltd., Hangzhou, China.

### Citrulline:

Citrulline (H₂N—C(O)—NH—(CH₂)₃—CH(NH₂)—CO₂H) is an alpha amino acid that has been found to relax blood vessels by its stimulation of nitric oxide production for endothelial function enhancement. In a recent study it was also found to reduce muscle fatigue. It has been promoted as a performance enhancing athletic dietary supplement.

An effective dose of citrulline ranges between 200-1000 mg/dose depending upon the indication. Citrulline can be purchased from NOW foods, Inc., Bloomingdale, Ill.

### Nattokinase:

Nattokinase (Enzyme Commission Number EC 3.4.21.62 and CAS Registry Number 9014-01-1) is a pro-fibrinolytic enzyme found in the Chinese vegetable cheese Natto. Natto is produced by fermenting soybeans. Nattokinase can be readily extracted and purified from Natto. Nattokinase has fibrinolytic or clot dissolving properties, can prevent the aggregation of red blood cells; and aids in decreasing blood viscosity. The use of nattokinase can aid the vascular distribution of nutrients. Nattokinase and methods for preparing nattokinase are further described in U.S. Patent Publication No. 2007/0116699 (Holsworth), which is incorporated herein by reference.

An effective dose of Nattokinase ranges between about 2000 and about 8000 fibrinolytic units (F.U.)/dose, depending upon the indication. Nattokinase can be purchased from Doctor's Best, Inc., San Clemente, Calif.

### Carnosine:

Carnosine (beta-alanyl-L-histidine) is a natural amino-acid that has a variety of functions. Carnosine is a potent antioxidant as well as an antioxidant enhancer. Carnosine also helps to chelate ionic metals, aid in flushing toxins from the body, boost the immune system, aid in the sensation of smell, is known to reduce and prevent cell damage caused by beta amyloid, and helps to prevent aberrant glycosylation. Such glycosylation includes the cross linking of proteins and DNA molecules caused by sugar aldehydes reacting with an amino acid group on a protein molecule, which creates Advance Glycation End-products (AGEs). The production of AGEs have been linked to the development of disease states such as arterial stiffening, cardiovascular disease, cataract development, neurological impairment, diabetes and the aging process. Carnosine prevents glycation and may also play a role in the disposal of glycated protein compounds.

An effective dose of L-carnosine ranges between 100-500 mg/dose depending upon the indication; however a total daily dose should not exceed 600 mg. L-carnosine can be purchased from Pure Bulk Nutrition, Inc., Myrtle Point, Oreg.

### Anserine:

Anserine is beta-alanyl-N-methylhistidine, a dipeptide that can be obtained from the skeletal muscle and brain of mammals. Anserine is commercially available, including as a nitrate salt (CAS No. 10030-52-1).

### Coffeeberry:

Coffeeberry is the berries of the coffee plant (Coffea arabica) after the coffee bean has been removed. The berries can be dried to provide a powder having little or no caffeine, for use in nutraceutical formulations. Coffeeberry is high in antioxidants called phenolic acids and also contains several plant nutrients. Although it contains a small amount of caffeine, the usual content in the suggested dose is about one tenth that in a single cup of tea. One gram of coffeeberry powder can provide as much anti-free radical power as 33 grams of blueberries (www.coffeeberry.org/good4u.htm). The polyphenols found in coffee berry can neutralize toxic oxygen free radicals, protect against oxidative stress, reduce the incidence of oxidative pathologies, and provide health benefits associated with glucose management, depression and anxiety.

Oxygen radical absorbent capacity (ORAC) is used in interpreting a dose of coffeeberry, which offers the most potent, orally administered antioxidant supplement available. Approximate 15000 ORAC are present in one gram of coffeeberry. An effective dose of coffeeberry ranges between 400-1200 mg/day with a generally accepted dose of 400 mg. Coffeeberry can be purchased from Twinlabs, Inc, American Fork, Utah, or New Chapter, Inc., Brattleboro, Vt.

### Cocoa Beans:

Beans of Theobroma cacao, herein referred to as cocoa beans, have long been considered to have beneficial effects on the cardiovascular system. Cocoa beans contain high amounts of the stimulant theobromin and lesser levels of caffeine. The low amount of cocoa bean in several of the compositions described below is not high enough to produce the deleterious side effects of large amounts of caffeine. The cocoa beans can be conveniently used in the formulation in the form of a powder, which can be weighed to provide an appropriate portion. Suitable portion sizes as a component of a formulation described herein include about 50 mg to about 500 mg, or about 100 mg, 200 mg, 300 mg, 400 mg, or 500 mg, per dosage unit of the formulation. One 400 mg dose of cocobean extract or powder provides 160 mg of polyphenols, which are phytochemicals with potent antioxidant properties.

Powder Compositions and Liquid Formulations Thereof

The compositions described herein can be provided in a liquid formulation or as a powder, which can be dissolved in a liquid of choice such as water, sparkling water, fruit juice, tea, coffee, hot chocolate, and the like. The composition containing nattokinase is typically provided in powder form to maintain potency, until the composition is ready to be consumed. Any potable liquid can be used to reconstitute the powder into a beverage.

The ingredients described herein can be used to prepare nutraceutical compositions. The compositions can be in the form of a powder, or the compositions can be a solution or suspension of the powder in a liquid, for example, water (e.g., pure water, spring water, artesian water, and the like), fruit juice, tea, coffee, hot chocolate, and the like.

The compositions described herein may be prepared in combination with inert diluents or edible carriers. For oral administration, as an alternative to preparing a drink or cocktail of the composition, compositions can be enclosed in hard or soft shell gelatin capsules, compressed into tablets, or incorporated directly into the food of a patient's diet. Compositions may be combined with one or more excipients and used in the form of ingestible tablets, troches, capsules, elixirs, suspensions, syrups, wafers, and the like. Such compositions and preparations typically contain at least 5% of a formulation described herein. The tablets, troches, pills, capsules, and the like may also contain one or more binders such as gum tragacanth, acacia, corn starch or gelatin; excipients such as dicalcium phosphate; a disintegrating agent such as corn starch, potato starch, alginic acid and the like; and/or a lubricant such as magnesium stearate.

Optional ingredients include flavoring agents such as peppermint, oil of wintergreen, or other natural flavors such as cherry, raspberry, grape, orange, banana, mango, or peach. When a unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, or shellac and the like. Coatings or separate packaging can be provided to ensure dryness of a component until the component is to be ingested or combined with other ingredients to form a beverage. Coatings or separate packaging can also be used to ensure chemical stability and to maintain the potency of a component. For example, arginine is hygroscopic and therefore should be kept dry until such time as a final formulation is to be prepared and consumed.

The formulation may be provided as a powder, syrup, or elixir. These preparations may optionally contain sucrose, fructose, or lactose as sweetening agents, methyl and propyl parabens as preservatives, coloring agents or dyes, and various suitable flavoring agents. Any material used in preparing any unit dosage form should be nutraceutically acceptable and non-toxic in the amounts employed. In some embodiments, synthetic sweeteners or components can be excluded. In other embodiments, a non-caloric artificial sweetener can be added for enhancing the taste of the formulation.

The powders and beverages described herein can also include one or more electrolytes and/or other additives. Electrolytes include sodium, potassium, magnesium, zinc, copper, chloride, and the like. Examples of suitable electrolytes include sodium chloride or sodium carbonate, potassium chloride or potassium carbonate, sodium chloride or potassium chloride, calcium chloride or calcium carbonate, magnesium chloride or magnesium carbonate, and other related electrolyte compounds. The electrolytes can be included in amounts of about 0.1 mg per dosage unit to about 50 mg per dosage unit.

Further optional additives include red yeast rice, and lipids and fatty acids present in fish oil, such as omega-3 fatty acids (e.g., a-linolenic acid (ALA), docosahexaenoic acid (DHA), eicosapentaenoic acid (EPA), and the like). The lipids can be oils or powders, such as the lipid powders available from ABF Ingredients Ltd., London, or Kerry Ingredients & Flavours, Beloit, Wis. Lipid powders can include up to about 73% lipid content of non-hydrogenated, trans-fat free, water dispersible oils in a convenient powdered form. The oil can be canola, high oleic sunflower, medium chain triglycerides, safflower, soybean, or sunflower oils, for example, dried on dairy or non-dairy bases. Suitable doses of fish oil or a component thereof include doses effective to maximize anti-platelet aggregation (e.g., 2-4 grams per day), or to reduce total cholesterol and enhance lipid profiles. Other optional additives include amino acids, proteins, preservatives, flavorings, colorings, and the like. Various additives can be included in amounts of about 0.1 mg per dosage unit to about 1000 mg per dosage unit.

The formulations, either as a powder or liquid containing the powder, can also be used to prepare an energy bar, such as a cereal bar or fruit and nut bar. The bar will preferably contain only natural ingredients and will not include excessive amounts of sucrose.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Formulations

In preparing the formulations described below, the following principles can be adhered to for providing advantageous compositions.

1. The formulations should contain at least one antioxidant and one energy compound.

2. It can be advantageous to add a vasodilator, a vasodilator adjuvant, or both.

3. Naturally isolated compounds can be preferred synthetic drugs.

4. Any compound, such as caffeine, with undesired side effects, can be omitted.

5. The ingredients can be chemically inert to each other, in that they do not react with each other prior to consumption.

6. No significant amount of refined sucrose, high fructose corn syrup or excessive sweetener of any kind will be included. Amounts of refined sucrose, high fructose corn syrup or excessive sweetener can be limited to less than 10 wt. %, 0.5-10 wt. %, less than 0.5 wt. %, or the components can be completely omitted.

7. A natural flavoring agent may be added, if desired. Some natural flavoring agents may contain a small amount of sweetener, which is acceptable.

8. The formulations should be palatable and have a pleasant taste and no significant aftertaste.

With these principles in mind, the following formulations can be prepared, as shown in the table below. For convenience, except for in the Summary and appended claims, the formulation refers to the mixture of ingredients without a liquid carrier. When the formulation is mixed with a liquid, it can be conveniently referred to as the composition.

Formulation

| Ingredient | 1 | | 2 | | 3 | | 4 | | 5 | | 6 | | 7 | |
|---|---|---|---|---|---|---|---|---|---|---|---|---|---|---|
| D-ribose | 3 | g | 1.5 | g | 3 | g | 3 | g | 3 | g | | | 3 | g |
| Guanosine | | | | | | | | | | | 1 | g | | |
| Citrulline | | | 500 | mg | 500 | mg | 500 | mg | 500 | mg | 500 | mg | 500 | mg |
| Nattokinase | | | | | 4000 | F.U. | 4000 | F.U. | 4000 | F.U. | 4000 | F.U. | 4000 | F.U. |
| Carnosine | 400 | mg | 400 | mg | | | 400 | mg | 400 | mg | 400 | mg | 400 | mg |
| Coffeeberry | | | | | | | | | 400 | mg | | | | |
| Cocoa bean | | | | | | | | | | | 200 | mg | 200 | mg |

A dose of the formulation powder can be mixed, dissolved, and/or suspended in a liquid for administration. The formulations in the table above were prepared and mixed with either 8 or 16 fluid ounces of water.

Each of these formulations may additionally include flavoring, carbonation, coloring, and/or electrolytes. Additional formulations may be prepared by varying the amount of ingredient in the formulation. For example, the amount of D-ribose can be about 0.5 g, 1 g, about 1.5 g, about 2 g, about 2.5 g, about 3 g, about 3.5 g, about 4 g, about 4.5 g, or about 5 g. The amount of carnosine can be about 100 mg, about 200 mg, about 300 mg, about 400 mg, or about 500 mg. The amount of citrulline can be about 300 mg, about 400 mg, about 500 mg, about 600 mg, about 700 mg, or about 750 mg. The amount of nattokinase can be about 1000 F.U., about 2000 F.U., about 3000 F.U., about 4000 F.U., about 5000 F.U., or about 6000 F.U. The amount of coffeeberry powder or extract can be about 100 mg, about 200 mg, about 300 mg, about 400 mg, or about 500 mg. The amount of cocoa bean powder can be about 100 mg, about 200 mg, or about 300 mg. The amount of guanosine can be about 1 g, about 1.5 g, about 2 g, about 2.5 g, about 3 g, about 3.5 g, about 4 g, about 4.5 g, or about 5 g.

In any of the above formulations, an additional energy compound, such as guanosine, creatine, and/or L-carnitine, can be added to the formulation, in addition to, or as a replacement for, D-ribose. An antioxidant, such as beta-carotene, coenzyme Q, selenium, vitamin C, and vitamin E, can also be added to any one of the above formulations. Other antioxidant enhancers, such as anserine, carnosine, coffeeberry powder, or cocoa bean extract, can also be added to any one of the above formulations, in place of, or in addition to, the carnosine, coffeeberry powder, or cocoa bean extract present in a particular formulation. Other vasodilators, such as L-arginine, adenosine, or xanthine, can be added to any one of the formulations above, for example, in addition to, or in place of, citrulline. Finally, a vasodilator adjuvant can be added to any of the formulations above. The vasodilator adjuvant can be, for example, aspirin, coumadin, or nattokinase.

Additionally, appropriate ratios of each ingredient to one or more other ingredients can be derived from the table above and also from the optional amounts above, to provide additional formulations of the invention having specific ratios of ingredients.

### Example 2

### Testing in Human Subjects

To test whether ingestion of the formulations of Example 1 improve energy and alertness, human volunteers were recruited. This pilot study, one week duration, involved three subjects: one 56 year old male and two females, 55 and 63 years of age. Following baseline assessments, each subject consumed Formulation 4 twice a day (AM and PM) with each dose of powder added to 6-8 ounces of water. Both subjective and objective effects were assessed (including completion of a questionnaire).

All subjects performed symptom limited incremental exercise with a 7.5 inch high step. Subjects increased their work load every minute by increasing step frequency. Breath by breath gas exchange was monitored using a chemical O₂ analyzer and infra-red CO₂ analyzer coupled with a differential pressure transducer. Objectively, minute ventilation (VE) in liters/min, tidal volume (TV) in mL per breath, heart rate (HR), partial pressure of end tidal CO₂, energy expenditure, VO₂, and VCO₂ were measured and functional status of the heart and lungs both at rest and during exercise were assessed. Heart rate recovery was also assessed during the first minute of recovery. Ventilation to perfusion matching in the lungs was also determined both at rest and during exercise using the ratio of mixed expired to end tidal CO₂. The anaerobic threshold (AT), or preferably the ventilator threshold (VT), was determined by the use of inspiratory drive, RER (resting energy expenditure) and VE/VO₂ profiles (equivalents of oxygen) during exercise. Based on the VT, O₂P (oxygen pulse) or SV (stroke volume) and tidal volume (TV) were determined. Vascular capacitance, the cross product of O₂P×ETCO₂ (end expiration of CO₂), was also determined at the AT.

The parameters described above are important in assessing cardiopulmonary-muscular efficiency as well as energy expenditure during exercise. Basal Metabolic Index (BMI) was measured and all three subjects were found to be within the normal range per gender, body height, and weight assessments.

In the following tables for Subject 1, Subject 2, and Subject 3:

Parameter 1: “HR @ VT” measures the heart rate at the determined ventilatory threshold, where “HR” is heart rate, and “VT” is the Ventilatory Threshold;

Parameter 2: “VO₂ @ VT”, measured in mL O₂ /kg/min, measures oxygen uptake at the determined ventilatory threshold, where “VO₂” is the volume of oxygen consumption in mL/kg/minute;

Parameter 3: “EE @ VT”, measured in Kcal/kg/hr, measures energy expenditure at the ventilatory threshold, where “EE” is energy expenditure;

Parameter 4: “O₂P×ETCO₂” (vascular capacitance or perfusion) measures perfusion blood volume during rest or exercise, where “O₂P” is oxygen pulse and “ETCO₂” is the end expired partial pressure of CO₂ in mm Hg;

Parameter 5: “V/Q ratio R/EX” measures the ratio of air flow to blood flow in lungs, where the “V/Q ratio” is the ventilation to perfusion ratio and “R/EX” is rest to exercise;

Parameter 6: “ETCO₂/HR” measures the partial pressure of CO₂ expired divided by the heart rate or the amount expired per heart beat;

Parameter 7: “HR to VO₂ slope” measures the coupling relationship between heart rate and oxygen uptake and is an indicator of autonomic tone or fitness, which can aid in identifying heart disease, where the slope is the linear regression of HR on the “y” axis plotted against VO₂ in mL/kg/min on the “x” axis;

Parameter 8: “OUES” is the oxygen uptake efficiency, slope, which measures O₂ uptake efficiency relative to ventilatory work. Its sub-components are lung, cardiac and peripheral perfusion factors and represents uptake of O₂ in the lungs, transport by the heart and extraction by the exercising muscle capillary beds;

Parameter 9: “O₂P @ VT”, measured in mL O₂ per beat, measures the oxygen pulse at the ventilatory threshold;

Parameter 10: “TV @ VT”, measured in mL ambient air, measures tidal volume attained at the ventilatory threshold, where “TV” is Tidal Volume; and

Parameter 11: “Peak ETCO₂”, measured in mm Hg, measures the partial pressure of exhaled CO₂ resulting from exercising tissue metabolism and buffering of lactate by bicarbonate.

The following tables summarize the cardiopulmonary exercise (CPX) Parameters.

Subject 1. KT: 56 Year Old Male with BMI=24.

| | | | | | Peak % |
|---|---|---|---|---|---|
| | | Baseline | Week | Week | Change from |
| # | CPX Parameter | Day 0 | 2 | 3 | Baseline |
| 1 | HR @ VT | 121 | 126 | 115 | +4% |
| 2 | VO₂ @ VT mL | 25 | 30 | 32.5 | +30% |
| | O₂/kg/min | | | | |
| 3 | EE @ VT | 8.35 | 9.69 | 9.47 | +16% |
| | Kcal/kg/hr | | | | |
| 4 | O₂P × ETCO₂ | 525 | 612 | 720 | +37% |
| 5 | V/Q ratio R/EX | .76/.87 | .69/.72 | .70/.86 | −7%/−17% |
| 6 | ETCO₂/HR | .33 | .33 | .36 | +9% |
| 7 | HR to VO₂ slope | 2.15 | 2.9 | 3.0 | +40% |
| 8 | OUES (O₂ uptake | 2.3 | 2.87 | 2.77 | +25% |
| | efficiency, slope) | | | | |
| 9 | O₂P @ VT mL O₂ | 15.0 | 17.0 | 20.0 | +33% |
| | per beat | | | | |
| 10 | TV @ VT in mL | 1150 | 1700 | 1650 | +48% |
| | ambient air | | | | |
| 11 | Peak ETCO₂ | 35 | 37 | 37 | +6% |
| | mm Hg | | | | |

Subject 2. JA: 55 Year Old Female with BMI=21 (previous smoking history).

| | | | | | Peak % |
|---|---|---|---|---|---|
| | | Baseline | Week | Week | change from |
| # | CPX Parameter | Day 0 | 2 | 3 | baseline |
| 1 | HR @ VT | 112 | 133 | 134 | +20% |
| 2 | VO₂ @ VT mL | 20 | 20 | 32 | +10% |
| | O₂/kg/min | | | | |
| 3 | EE @ VT | 5.5 | 7.14 | 7.1 | +30% |
| | Kcal/kg/hr | | | | |
| 4 | O₂P × ETCO₂ | 344 | 360 | 378 | +10% |
| 5 | V/Q ratio R/EX | .57/.56 | .62/.53 | .58/.52 | +9%/−5% |
| 6 | ETCO₂/HR | .38 | .30 | .34 | −11% |
| 7 | HR to VO₂ slope | 3.3 | 3.7 | 3.0 | +12% |
| 8 | OUES | 1.4 | 1.7 | 1.4 | +21% |
| 9 | O₂P @ VT mL O₂ | 8.4 | 7.2 | 8.5 | +1% |
| | per beat | | | | |
| 10 | TV @ VT in mL | 800 | 820 | 900 | +12.5% |
| | ambient air | | | | |
| 11 | Peak ETCO₂ | 43 | 41 | 42 | −2% |
| | mm Hg | | | | |

Subject 3. JH: 63 Year Old Female with BMI 22.

| | | | | | Peak % |
|---|---|---|---|---|---|
| | | Baseline | Week | Week | Change from |
| # | CPX Parameter | Day 0 | 2 | 3 | baseline |
| 1 | HR @ VT | 129 | 130 | 133 | +3% |
| 2 | VO₂ @ VT mL | 12.9 | 18.0 | 18.0 | +40% |
| | O₂/kg/min | | | | |
| 3 | EE @ VT | 5.10 | 5.24 | 5.65 | +11% |
| | Kcal/kg/hr | | | | |
| 4 | O₂P × ETCO₂ | 292 | 329 | 423 | +45% |
| 5 | V/Q ratio R/EX | .67/.71 | .74/.79 | .66/.68 | +10% to −11% |
| 6 | ETCO₂/HR | .36 | .36 | .38 | +6% |
| 7 | HR to VO₂ slope | 4.14 | 3.4 | 3.8 | −18% |
| 8 | OUES | 1.3 | 1.3 | 1.0 | −23% |
| 9 | O₂P @ VT mL O₂ | 6.8 | 8.0 | 8.4 | +24% |
| | per beat | | | | |
| 10 | TV @ VT mL | 867 | 910 | 1000 | +15% |
| | ambient air | | | | |
| 11 | Peak ETCO₂ | 45 | 47 | 47 | +4% |
| | mm Hg | | | | |

The tables above provide objective data for three subjects, measured at baseline and after two and three weeks. The parameters assessed in the tables indicate that administration of Formulation 4 in water for three weeks show the benefit of this supplement in increasing tissue perfusion by vasodilation (OUES, peak ETCO₂, and O₂P×ETCO₂ (vascular capacitance), VO₂ @ VT, and EE @ VT), e.g, from inclusion of citrulline, aiding in decreasing blood viscosity (OUES, O₂P, peak ETCO₂, O₂P×ETCO₂, VO₂ @ VT, and EE @ VT), e.g., from inclusion of nattokinase, as well as providing ATP levels to meet demand at the tissue level during exercise (OUES, O₂P, peak ETCO₂, O₂P x ETCO₂, VO₂ @ VT, and EE @ VT), e.g., from inclusion of D-ribose.

There was a significant increase in VO₂, energy expenditure, O₂P, ventilatory threshold, and vascular capacitance at the ventilatory threshold in each subject. The observed improvements in these parameters indicate that this formulation improves tissue perfusion and the release of oxygen to muscle beds during exercise. The formulation also provides an improved energy state or vitality of both the heart and lungs to perform more efficiently. Also noteworthy is that there was also significant improvement in O₂ uptake efficiency even after only two weeks of consumption. Oxygen uptake efficiency, which directly correlates to peak or maximum attained VO₂, is assessed by the linear slope of oxygen uptake to the log of VE or minute ventilation. Increased OUES reflects increased uptake in the lungs, transport by the heart and uptake in the exercising muscle.

These “triple action” physiological and biochemical properties from the components of the formulation result in a improvements in lung perfusion and subsequent function, increases in O₂ delivery via reduced red blood cell viscosity, and vascular vasodilation and increased cardiac output. This novel formulation also provides enhanced antioxidant effects, increased production of nitric oxide for endothelial cell function, and increases the efficiency of cardiopulmonary coupling with an ideal adenine nucleotide substrate for the production of ATP during exercise, where cellular energy production and unloading of oxygen at the tissue level is crucial.

High intensity exercise can deplete high energy compounds, leading to a state where demand exceeds supply. The beneficial qualities from each component in this supplement aids the heart, lungs, skeletal muscle, and peripheral vasculature to help in maintaining a metabolic balance during times of stress, including exercise, as demonstrated in this study.

As discussed above, the energy benefits seen in this medium-level sub-maximal stress exercise also apply to persons undergoing vigorous exercise, and those at rest. The compositions are especially beneficial to aging populations, for example, those who are unable to exercise due to inanition or desire not to participate in a regular structured exercise regimen.

### Example 3

### Subjective Measures

The improvement in cardio-pulmonary status shown in Example 2 indicates that persons taking the formulation experienced less fatigue and improved metabolic balance or homeostasis. They also reported better sense of well-being. In order to confirm these assessments subjectively, participants were asked to complete the following questionnaire, giving a numerical estimate to each of the following questions.

1. How is your stamina in the morning? 1=low; 5=moderate; 10=great.

2. How is your stamina in the late afternoon? 1=low; 5=moderate; 10=great.

3. What is your level of fatigue? 1=constantly tired; 5=moderately tired; 10=never tired.

4. What is your typical sleep pattern? 1=difficult falling asleep; 5=restless; 10=sleep soundly.

5. How is your mental clarity? 1=difficulty concentrating; 5=easily distracted; 10=high.

6. What is your level of daily perceived pain? 1=high level of pain; 5=moderate to minimal pain; 10=no pain.

7. What is your level of well-being? 1=poor; 5=moderate; 10=very good.

The responses of the three participants of Example 2 are summarized in the following table. For the heading “Stamina, Morning to Afternoon” (a comparison of Questions 1 and 2 above), the numbers are the changes from morning to afternoon (“AM value”-“PM value”) at baseline (day 0), after the second week, and after the third week, respectively. The values listed for Questions 3-6 are also listed for assessments at baseline (day 0), after the second week, and after the third week, respectively.

| | Question: | | | | |
|---|---|---|---|---|---|
| | 1 and 2 | | | | |
| | Stamina, | 3 | 4 | 5 | 6 |
| | Morning to | Fatigue | Sleep | Mental | Perceived |
| Subject | Afternoon | Level | Pattern | Clarity | Pain |
| 1 | 4-2; 5-7; 6-5 | 5; 7; 7 | 4; 5; 7 | 3; 6; 8 | 7; 8; 9 |
| 2 | 10-5; 10-8; 10-8 | 5; 8; 8 | 5; 8; 8 | 6; 8; 8 | 9; 5*; 5* |
| | | | | | *injured |
| | | | | | ankle |
| 3 | 7-5; 1-9; 10-10 | 6; 9; 9 | 9; 10; 10 | 7; 9; 10 | 7; 9; 10 |

As can be determined by the data in the table, all three subjects improved in most of the categories and had achieved near-maximum benefit by the second week. Beyond these generalities, each participant was unique depending on his or her initial state, metabolism and other factors that were uncontrolled in this subjective study, but consumption of Formulation 4 clearly benefited all three subjects.

Subject 1 had, in general, the lowest evaluations for the baseline values. His maintenance of stamina through the day and his mental clarity were the most striking improvements. Overall, his level of well-being went from 5 to 7.

Subject 2 had moderate improvement across the board. Perhaps because of an optimistic personality, she rated her level of well-being as very good from baseline to week three, despite pain from her ankle injury (well-being score of 10 throughout).

Subject 3 is particularly interesting in having raised her morning stamina from a moderate 5 to a great 10 by the second week. Again, her initial level of well-being was a high, 9, which increased to 10 by week two.

These results show that the objective assessments (Example 2) match the states that can be perceived by persons taking these supplements, as illustrated here in this assessment.

### Example 4

### Measurement of Urinary Metabolites of Reactive Oxygen Species (ROS)

Reactive oxygen species (ROS) have very short half lives. It is therefore difficult or impossible to measure ROS in the blood or tissues. However, certain metabolites of ROS can be found in the blood and are excreted in the urine. It is well accepted that these metabolites are a reliable measure of the production of ROS in the tissues. One such metabolite, malondialdehyde (MDA) is particularly useful as a marker of ROS production. An MDA analysis kit can be purchased from Oxis Research, Portland, Oregon.

The formulations described herein will be administered to volunteers for defined periods of time, followed by assessment of their MDA levels. Inclusion criteria will include:
Present complaint of fatigue for over one month;
Males/females, 30-70 years of age;
No previous history of cardiac, pulmonary or metabolic disease;
Capable of performing sub-maximal exercise testing;
Normal blood pressure;
Understands the required daily routine, including the formulation consumption;
Can meet assessment and evaluation requirements; and
Understands and signs a consent form.

Persons will not be enrolled in this study if they take any dietary supplement, except for multi-vitamins, or have a history of diseases including cardiac, thyroid, metabolic conditions. They must be able to cooperate and comply with the protocol and be able to document the requested data. Persons who are known to be noncompliant will also be excluded.

The volunteers will be seen in a clinic where they will perform sub-maximal exercise as described in Example 1. As in that study, the volunteers will take Formulation 4, twice daily, throughout a three week period. At the clinic visits, urine and/or serum samples will be drawn for MDA analysis and each participant will monitor their perceived fatigue level according to a questionnaire provided to them.

A series of questions will assist in their self-evaluation, such as “how is your stamina in the morning and late afternoon”, and questions regarding sleep history, mental clarity, daily perceived pain, and overall sense of well-being. The subjects will be asked to complete this questionnaire at baseline (day 0), at the end of week 2, and at the end of week 3. At these points, urine and/or serum samples will be drawn to determine the production of ROS during sub-maximal exercise.

Based on the results of Examples 2 and 3 above, it is expected that supplementation with the formulations described herein will decrease the production of ROS metabolites, indicating a reduction in the production of ROS in the subjects taking the formulations.

### Example 5

### Prevention of Deleterious Effects of ROS on Immune Function

It has long been known that stress impairs immune function. With the more recent research on ROS, it is now known that this impairment is mediated through the stress-induced production of neuroendocrine hormones, particularly glucocorticoids and catecholamines, and to a lesser extent, prolactin, growth hormone, and nerve growth factor. Through the action of these stress-induced hormones, detrimental effects have been shown on immune function, including reduced NK (natural killer) cell activity, reduced lymphocyte populations, reduced antibody production, and reactivation of latent viral infections.

Based on the results of Examples 2, 3, and 4, it is expected that the deleterious effects of stress on immune function will be reduced by regular consumption of one or more of the formulations described herein.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A composition comprising an energy compound and at least one of a vasodilator, a vasodilator adjuvant, and an antioxidant enhancer;
wherein the energy compound comprises D-ribose or guanosine.

2. The composition of claim 1 wherein the antioxidant enhancer is carnosine.

3. The composition of claim 2 wherein the vasodilator is citrulline.

4. The composition of claim 3 wherein the vasodilator adjuvant is nattokinase.

5. The composition of claim 4 further comprising one or two additional antioxidant enhancers.

6. The composition of claim 5 wherein the additional antioxidant enhancer is one or both of coffeeberry powder and cocoa bean powder.

7. The composition of claim 6 further comprising one or more antioxidants.

8. The composition of claim 7 wherein the antioxidant is beta-carotene, coenzyme Q, selenium, vitamin C, vitamin E, acai, quercetin, pomegranate, astaxanthin, or a combination thereof

9. The composition of claim 1 further comprising a lipid based powder.

10. The composition of claim 1 wherein the wt. % ratio of the energy compound to the antioxidant enhancer is about 1.6:1 to about 7.5:1.

11. The composition of claim 1 wherein the wt. % ratio of the energy compound to the vasodilator is about 2:1 to about 6:1.

12. The composition of claim 1 further comprising water.

13. A composition comprising a) D-ribose or guanosine; b) citrulline or L-arginine; c) nattokinase; and d) carnosine.

14. The composition of claim 13 further comprising coffee berry powder, cocoa bean powder, or both.

15. The composition of claim 14 further comprising one or more antioxidants.

16. A method to treat oxidative stress comprising administering to a human experiencing oxidative stress an effective amount of a composition of claim 1, thereby reducing the amount of oxidative stress experienced by the human.

17. A method to increase tissue perfusion comprising administering to a human in need of increased tissue perfusion an effective amount of a composition of claim 1, thereby increasing the tissue perfusion in the human.

18. A method to increase tissue perfusion comprising administering to a human in need of increased tissue perfusion an effective amount of a composition of claim 13, thereby increasing the tissue perfusion in the human.

19. A method to aid aerobic work performance comprising administering to a human engaging in aerobic work an effective amount of a composition of claim 1, thereby increasing the aerobic work capacity of the human.

20. A method to improve blood viscosity comprising administering to a human an effective amount of a composition of claim 1, thereby reducing the viscosity of the blood in the human.
